(19) United States
(12) Patent Application Publication
(10) Pub. No.: US 2026/0266941 A1
(43) Pub. Date: Sep. 10, 2026

(54) MAGNETIC RESONANCE IMAGING METHOD AND MAGNETIC RESONANCE IMAGING SYSTEM

(71) Applicant: GE Precision Healthcare LLC, Waukesha, WI (US)

(72) Inventors: Yao Ding, Beijing (CN); Yongchuan Lai, Beijing (CN); Xiaocheng Wei, Beijing (CN)

(21) Appl. No.: 19/560,738

(22) Filed: Mar. 9, 2026

(30) Foreign Application Priority Data
Mar. 7, 2025 (CN) 202510276092.4

(51) Int. Cl. G01R 33/567; A61B 5/00

(52) U.S. Cl. CPC G01R 33/5673; A61B 5/7285

(57) ABSTRACT

Examples of the present application provide a magnetic resonance imaging method and a magnetic resonance imaging system. The method includes: triggering, in one cardiac cycle or each of a plurality of consecutive cardiac cycles, after a preceding specific cardiac signal is detected, a pulse sequence for acquiring a magnetic resonance signal; acquiring a time length from the end of the pulse sequence to a succeeding cardiac signal first detected after the end of the pulse sequence; determining, on the basis of the time length, whether the magnetic resonance signal acquired on the basis of the pulse sequence is abnormal; and processing the abnormal magnetic resonance signal.

### CROSS-REFERENCE TO RELATED APPLICATIONS

The present application claims priority to and benefit of Chinese Application No. 202510276092.4, filed on Mar. 7, 2025, which is incorporated by reference in its entirety.

### TECHNICAL FIELD

Embodiments of the present application relate to the technical field of medical devices, and in particular to a magnetic resonance imaging method and a magnetic resonance imaging system.

### BACKGROUND

Cardiac magnetic resonance imaging (cardiac MRI, CMR) is non-invasive medical imaging technology that is widely used in the diagnosis and evaluation of heart diseases. To avoid the impact of cardiac motion on image quality, a cardiac gating technique is typically used to synchronize cardiac motion with magnetic resonance image acquisition. Magnetic resonance imaging systems can access electrocardiogram signals detected by external electrocardiogram devices, and control, in conjunction with the electrocardiogram signals, magnetic resonance scans or process image data acquired via the magnetic resonance scans. In this way, for example, sufficient images are allowed to be acquired during the late diastole, thereby reducing motion artifacts.

However, in reality, the cardiac motion of many subjects under examination, who may have, for example, arrhythmia, is not uniform. In such cases, image data acquired by traditional cardiac imaging methods may not all be data from the late diastole, but may also include data from other states of the heart such as the systolic state, which may introduce motion artifacts and increase the complexity of image processing.

### SUMMARY OF THE INVENTION

According to an aspect of embodiments of the present application, a magnetic resonance imaging method is provided. The method includes: triggering, in one cardiac cycle or each of a plurality of consecutive cardiac cycles, after a preceding specific cardiac signal is detected, a pulse sequence for acquiring a magnetic resonance signal; acquiring a time length from the end of the pulse sequence to a succeeding cardiac signal first detected after the end of the pulse sequence; determining, on the basis of the time length, whether the magnetic resonance signal acquired on the basis of the pulse sequence is abnormal; and processing the abnormal magnetic resonance signal.

According to an aspect of the embodiments of the present application, a magnetic resonance imaging system is provided, the system comprising: a scanning unit; and a controller, configured to control the scanning unit to acquire a magnetic resonance signal and perform the foregoing magnetic resonance imaging method.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a magnetic resonance imaging system according to embodiments of the present application;

FIG. 2 is a schematic diagram of a gating method used in existing magnetic resonance imaging technology;

FIG. 3 is a flowchart of a magnetic resonance imaging method according to an embodiment of the present application;

FIG. 4 is a schematic timing diagram of the embodiment shown in FIG. 3;

FIG. 5 is a flowchart of a magnetic resonance imaging method according to another embodiment of the present application;

FIG. 6 is a schematic timing diagram of the embodiment shown in FIG. 5;

FIG. 7 is a timing diagram used for a magnetic resonance imaging method according to another embodiment of the present application;

FIG. 8 is a flowchart of a magnetic resonance imaging method according to another embodiment of the present application;

FIG. 9 is an example of a timing diagram of the embodiment shown in FIG. 8;

FIG. 10 is another example of a timing diagram of the embodiment shown in FIG. 8; and

FIG. 11 shows images obtained by performing magnetic resonance scans on a subject with arrhythmia using an embodiment of the present application and the prior art.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements by their title, but do not represent a spatial arrangement or temporal order, etc., of these elements, and these elements should not be limited by these terms. The term “and/or” includes any one of and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise explicitly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

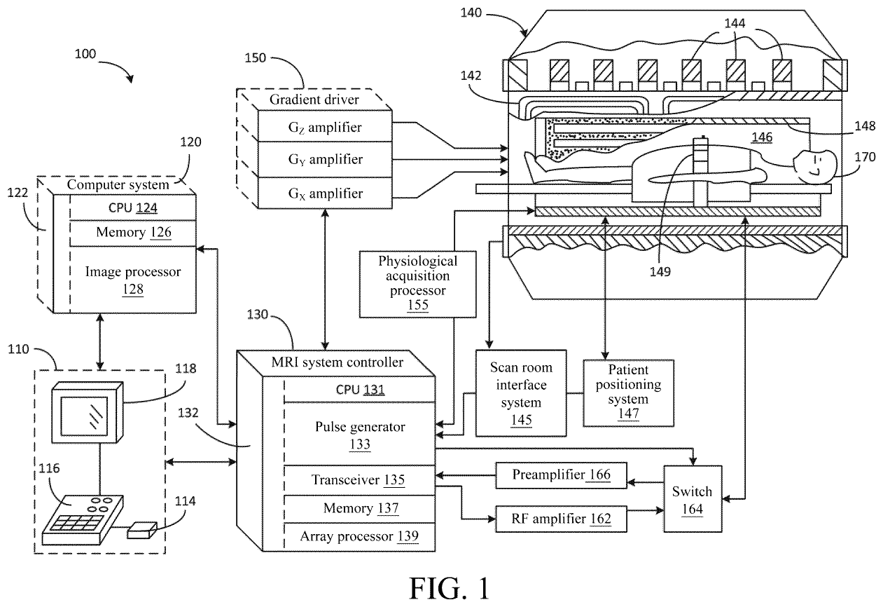

For ease of understanding, FIG. 1 shows a magnetic resonance imaging (MRI) system 100 according to some embodiments of the present application.

The MRI system 100 includes a scanning unit 140. A subject or patient 170 of an MRI scan may be located in the scanning unit 140, specifically within a cylindrical imaging volume 146. The scanning unit 140 is used to perform a magnetic resonance scan on the subject 170 to generate image data of a region of interest of the subject 170. The region of interest may be a predetermined anatomical site or anatomical tissue.

The scanning unit 140 includes a superconducting magnet having a superconducting coil 144 that, in operation, provides a static uniform longitudinal magnetic field B₀ throughout the cylindrical imaging volume 146. The scanning unit 140 further includes a gradient coil assembly 142 and an RF body coil 148. The RF body coil 148 provides a transverse magnetic field B₁ in operation. The transverse magnetic field B₁ is approximately perpendicular to B₀ in the entire cylindrical imaging volume 146, and is used to excite excited nuclei in the body of the subject 170 to generate an MR signal. The gradient coil assembly 142 may be used to provide gradient magnetic fields in, for example, three directions (X, Y, and Z) for superposition on a B₀ field, and provide position encoding of the MR signal. The scanning unit 140 may further include an RF surface coil 149. The RF body coil 148 and the RF surface coil 149 may be configured to operate in a transmit and receive mode, a transmit mode, or a receive mode.

Operation of the MRI system 100 is controlled by an operator workstation 110, and the operator workstation 110 includes an input device 114, a control panel 116, and a display 118. The input device 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and communicates with a computer system 120, and the computer system enables an operator to control the generation and viewing of an image on the display 118. The computer system 120 includes a plurality of components that communicate with one another by means of an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced with an image processing function implemented in the CPU 124. The computer system 120 may be connected to an archival media device, a persistent or backup memory, or a network. The computer system 120 may be coupled to and communicate with a separate MRI system controller 130.

The MRI system controller 130 includes a set of components that communicate with one another by means of an electrical and/or data connection module 132. The connection module 132 may employ a direct wired connection, a fiber optic connection, a wireless communication link, etc. The MRI system controller 130 may include a CPU 131, a pulse generator 133 that communicates with the operator workstation 110, a transceiver (or an RF transceiver) 135, a memory 137, and an array processor 139. In some embodiments, the pulse generator 133 may be integrated into the scanning unit 140 of the MRI system 100. The MRI system controller 130 can receive a command from the operator workstation 110 to indicate the pulse sequence to be executed during an MRI scan, so as to control the scanning unit 140 to perform the aforementioned magnetic resonance scan procedure.

The aforementioned “pulse sequence” (also known as a scan sequence or an imaging sequence) refers to a combination of pulses with specific amplitudes, widths, directions, and timing applied during a magnetic resonance scan. These pulses may typically include, for example, a radio frequency pulse and a gradient pulse. The radio frequency pulse may include, for example, a radio frequency excitation pulse, a radio frequency refocusing pulse, a preparation pulse, etc. The gradient pulse may include, for example, a gradient pulse used for layer selection, a gradient pulse used for phase encoding, a gradient pulse used for frequency encoding, a gradient pulse used for phase shifting (phase shift), a gradient pulse used for phase dispersion (dephasing), etc.

Typically, a plurality of pulse sequences may be preset in the magnetic resonance system, so that a sequence suitable for clinical examination requirements can be selected. The clinical examination requirements may include, for example, an imaging site, an imaging function, an imaging effect, etc.

Specifically, the pulse generator 133 in the MRI system controller 130 generates a low-power radio frequency signal and sends the low-power radio frequency signal to an RF amplifier 162 via the transceiver 135. The RF amplifier 162 amplifies the low-power radio frequency signal and provides the amplified low-power radio frequency signal to the RF body coil 148 via a transmit/receive switch (T/R switch) 164. The RF body coil 148 then provides the transverse magnetic field B₁. The transverse magnetic field B₁ is approximately perpendicular to B₀ in the entire cylindrical imaging volume 146.

The MRI system controller 130 provides a gradient waveform to a gradient driver 150. The gradient driver system includes Gₓ (X direction), G_{y} (Y direction), and G_{z} (Z-direction) amplifiers, etc. Each of the Gₓ, G_{y}, and G_{z} gradient amplifiers excites a corresponding gradient coil in the gradient coil assembly 142, to generate a gradient magnetic field used to spatially encode an MR signal during an MRI scan. The X direction may also be referred to as a frequency encoding direction or a kₓ direction in k-space. The Y direction may be referred to as a phase encoding direction or a k_{y} direction in the k-space. Gₓ may be used for frequency encoding or signal readout, and is generally referred to as a frequency encoding gradient or a readout gradient. G_{y} may be used for phase encoding, and is generally referred to as a phase encoding gradient. G_{z} may be used for slice (layer) position selection to obtain k-space data. It should be noted that a layer selection direction, a phase encoding direction, and a frequency encoding direction may be modified according to actual requirements.

In some embodiments, the MRI system controller 130 is coupled to a scanning chamber interface system 145 and communicates therewith, and the scanning chamber interface system 145 receives signals from various sensors associated with a state of the scanning unit 140. The scan room interface system 145 is further coupled to and communicates with a patient positioning system 147, and the patient positioning system sends and receives signals to control the movement of a patient table to a desired position to perform the MRI scan.

In some clinical applications, a gating triggering technique is typically utilized to trigger magnetic resonance signal acquisition, such as repeatedly triggering a selected pulse sequence. This may require real-time monitoring of vital signs such as respiration or heartbeat, generation of a gating signal when a specific vital sign signal is detected, and triggering the MRI system controller 130 to send a pulse sequence to obtain a magnetic resonance signal with a high signal-to-noise ratio. In the case of cardiac magnetic resonance imaging, for example, in order to eliminate artifacts, electrocardiogram techniques may be used to control the time of data acquisition within a relatively motionless or stable period of the heartbeat cycle, such as at the mid-to-late diastole of the cardiac cycle, to perform magnetic resonance acquisition.

In an embodiment of the present application, the MRI system controller 130 may also receive data from a physiological acquisition controller 155, which receives signals from a plurality of different sensors (such as electrocardiogram (ECG) signals from electrodes attached to a patient) connected to the subject 170 to acquire physiological signals of the subject 170. For example, one or more of these sensors are used to acquire heartbeat signals (also referred to as cardiac signals) of the subject 170.

In an embodiment of the present application, the physiological acquisition processor 155 may receive detected heartbeat signals and generate, on the basis of the detected heartbeat signals, a cardiac waveform, which may include, for example, an R wave. The physiological acquisition processor 155 or the system controller 130 communicating therewith may further determine a trigger node of a pulse sequence on the basis of the waveform. In some embodiments, the physiological acquisition processor 155 may be integrated into the MRI system controller 130.

As described above, excited nuclei within the subject 170 located in a selected magnetic field region (which is determined, for example, by a gradient magnetic field) are excited by a radio frequency pulse to generate an MR signal. The MR signal may be sensed and received by the RF body coil 148 or the surface coil 149 and sent back to a preamplifier 166 via the switch 164. The switch 164 may be controlled by a signal from the pulse generator 133 to electrically connect the RF amplifier 162 to the RF body coil 148 in a transmit mode, and connect the preamplifier 166 to the RF body coil 148 or the surface coil 149 in a receive mode.

In some embodiments, the MR signals sensed and received by the RF body coil 148 or the RF surface coil 149 and amplified by the pre-amplifier 166 are demodulated, filtered, and digitized in a receiving portion of the transceiver 135, and transmitted to the memory 137 in the MRI system controller 130. For each image that is to be reconstructed, the data is rearranged into a separate k-space data array, each of the separate k-space data arrays is inputted into the array processor 139, and the array processor is operated to transform the data into an array of image data by means of a Fourier transform.

The array processor 139 uses a transform method, most commonly a Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems.

The MRI system controller 130, the image processor 128, and the physiological acquisition processor 155 may separately or collectively include a computer processor and a storage medium. The storage medium records a predetermined data processing program that is to be executed by the computer processor. For example, the storage medium may store a program used to implement scanning processing (for example, a scan procedure and an imaging sequence), image reconstruction, image processing, etc. For example, the storage medium may store a program used to implement the magnetic resonance imaging method according to the embodiments of the present application. The above storage medium may include, for example, a ROM, a floppy disk, a hard disk, an optical disk, a magneto-optical disk, a CD-ROM, or a non-volatile memory card.

It should be understood that the MRI system 100 shown in FIG. 1 is intended for illustration. A suitable MRI system may include more, fewer, and/or different components.

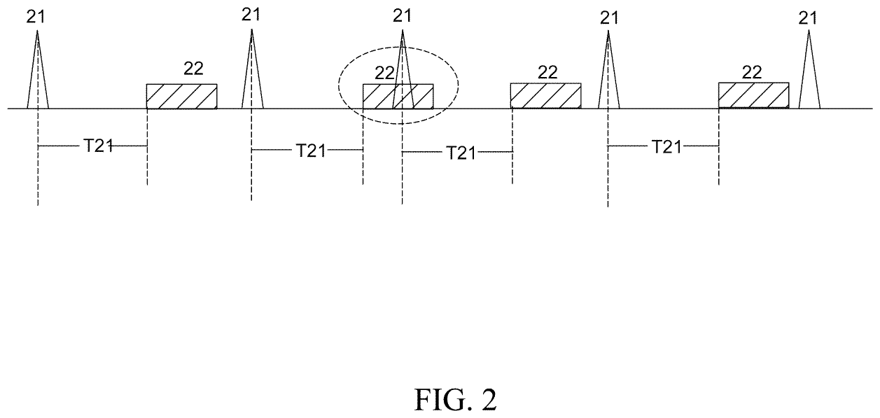

As mentioned above, gating techniques are used to address image quality issues caused by uneven heartbeat motions, which include a prospective gating technique and a retrospective gating technique. In the prospective gating technique, a pulse sequence is triggered on the basis of a specific cardiac signal, which usually corresponds to an R wave in an electrocardiogram waveform. FIG. 2 shows a schematic diagram of a gating method used in existing magnetic resonance imaging technology, in which a pulse sequence 22 is triggered after an R wave 21 according to a preset delay T21, which allows a cardiac image when the heart is relatively motionless to be acquired, and allows images acquired in a plurality of time periods to correspond to substantially the same phase. However, in cases of cardiac arrhythmia, the lengths of different R-wave-based time periods vary, which introduces motion artifacts. For example, as shown by a circular dashed line in FIG. 2, when a distance between adjacent R waves 21 is overly short, a pulse sequence 22 triggered by a previous R wave 21 may occur when a next R wave 22 occurs, and thus a magnetic resonance image acquired on the basis of the pulse sequence 22 corresponds to cardiac systole. To address this issue, the magnetic resonance signal acquired on the basis of the pulse sequence 22 may be discarded during subsequent data processing, while magnetic resonance signals generated by other pulse sequences not applied during the R-wave occurrence are retained.

The inventors of the present application further discovered that when a time period (e.g., an R-R interval) in which a pulse sequence is located is overly long, although the pulse sequence is not executed during cardiac systole, there is still an artifact issue caused by phase inconsistency. Furthermore, when a preparation pulse is further applied before the pulse sequence, an additional artifact issue could result from an overly long distance between R waves.

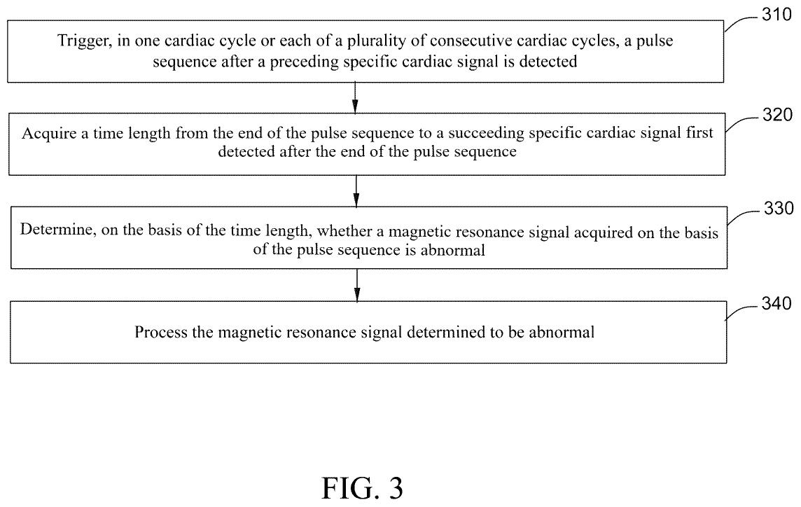

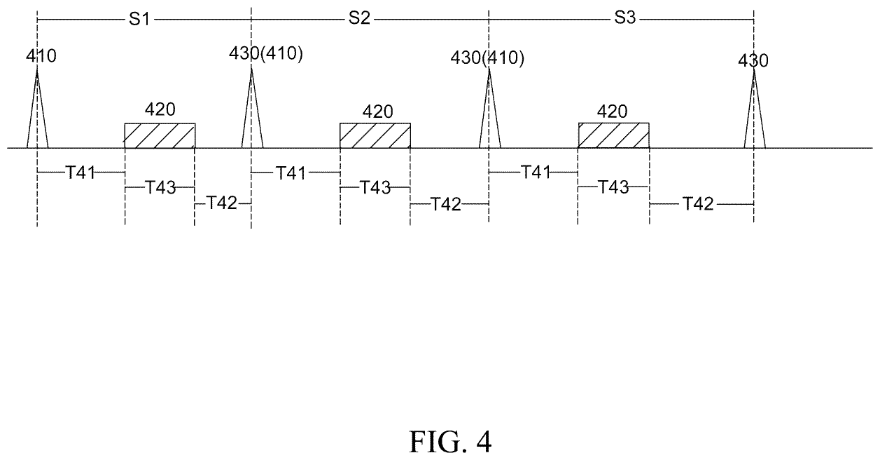

FIG. 3 is a flowchart of a magnetic resonance imaging method according to an embodiment of the present application, and FIG. 4 is a schematic timing diagram of the embodiment shown in FIG. 3. Referring to FIG. 3 and FIG. 4, in step 310, in one cardiac cycle (e.g., any one of cardiac cycles S1, S2, and S3 in FIG. 4) or in each of a plurality of consecutive cardiac cycles (e.g., cardiac cycles S1, S2, and S3), a pulse sequence 420 is triggered after a preceding specific cardiac signal 410 is detected. As described above, the pulse sequence 420 is for acquiring a magnetic resonance signal. The signal acquisition here does not narrowly refer to acquiring signals by a receive coil, but includes a process of transmitting a sequence for signal acquisition and acquiring signals. Since the cardiac motion is periodic, the specific cardiac signal can usually be detected repeatedly. The preceding specific cardiac signal 410 may be any one of a plurality of specific cardiac signals that are detected repeatedly in a plurality of cardiac cycles. Time between two adjacent specific cardiac signals may be referred to as a cardiac cycle. Those skilled in the art will understand that the pulse sequence may be predetermined by an operator and suitable for a specific clinical application (e.g., cardiac imaging). The pulse sequence may be a repetition time (TR) of a complete pulse sequence, or a part of that repetition time. Those skilled in the art will understand that the repetition time refers to a time interval between two adjacent radio frequency excitation pulses in a complete pulse sequence. The repetition time has a preset duration, and each repetition time has the same pulse timing. In this step, a repetition time of the pulse sequence may be triggered on the basis of the detected preceding specific cardiac signal 410. The aforementioned specific cardiac signal may be a signal with specific characteristics among detected periodic cardiac signals, such as an R wave of an electrocardiogram waveform. Depending on different clinical needs, the specific cardiac signal may also be other signals that can represent the state of cardiac motion.

The aforementioned “trigger” may include, for example, sending pulse signals by a system controller of a magnetic resonance system to radio frequency, gradient, and other magnetic resonance subsystems according to preset timing after a first preset delay time T41 following the specific cardiac signal 410. The first preset delay time T41 may be determined on the basis of time required from the end of the specific cardiac signal to an expected heartbeat state (e.g., mid-to-late diastole) in relatively normal heartbeat cycles (or under normal circumstances), and the first preset delay time T41 is equal in each cardiac cycle.

In step 320, a time length T42 from the end of the pulse sequence 420 to a succeeding specific cardiac signal 430 first detected after the end of the pulse sequence 420 is acquired. For ease of understanding and distinction, “preceding” and “succeeding” are used only to indicate a relative order of specific cardiac signals that can be detected repeatedly or a relative order of cardiac cycles. Furthermore, the succeeding specific cardiac signal 430 may be used as a preceding specific cardiac signal 410 (denoted as “430 (410)” in the figure) in a succeeding cardiac cycle. For example, a succeeding specific cardiac signal 430 in the cardiac cycle S1 is used as a preceding specific cardiac signal 410 in the cardiac cycle S2, and so on. In the embodiments of the present application, by acquiring the time length between the end of the pulse sequence and the succeeding specific cardiac signal first detected after the end of the pulse sequence, it can be determined whether a cardiac cycle (or time period) between the two specific cardiac signals is overly long or overly short.

In step 330, on the basis of the acquired time length T42, it is determined whether the magnetic resonance signal acquired on the basis of the pulse sequence is abnormal.

In step 340, the magnetic resonance signal that is determined to be abnormal is processed. In some embodiments, the processing includes discarding or correcting the magnetic resonance signal acquired via the corresponding pulse sequence. Therefore, the embodiments of the present application can eliminate or improve abnormal magnetic resonance data caused by overly long or short cardiac cycles in a timely manner, thereby avoiding artifact issues caused by the abnormal data.

In some embodiments, a threshold may be set on the basis of analysis of heartbeat motion of a single subject or a plurality of subjects in a specific population. This specific population may be determined on the basis of gender, age, and disease type, for example. The aforementioned threshold may include, for example, a first preset value, which may represent a large time length. When the time length T42 between the end of the pulse sequence and the detection of the succeeding specific cardiac signal 430 is greater than the first preset value, it indicates that the current cardiac cycle (e.g., the cardiac cycle S3) of the subject is overly long, and the magnetic resonance signal acquired via the corresponding pulse sequence (the magnetic resonance signal acquired at the cardiac cycle S3) may not be used as image data for the heartbeat of the subject under ideal clinical conditions.

Similarly, the threshold may further include a second preset value, which may be a small threshold for the time length. When the time length (e.g., the time length T42 at the cardiac cycle S1) is less than the second preset value, or when another specific cardiac signal is detected during the pulse sequence unexpectedly (e.g., when the first R wave appears before the pulse sequence ends despite normal circumstances in which the pulse sequence ends before the first R wave arrives after the pulse sequence begins), the corresponding magnetic resonance signal is determined to be abnormal.

The first preset value and the second preset value mentioned above may be upper and lower bounds of a range of values for this time length determined on the basis of the normal cardiac cycle, respectively.

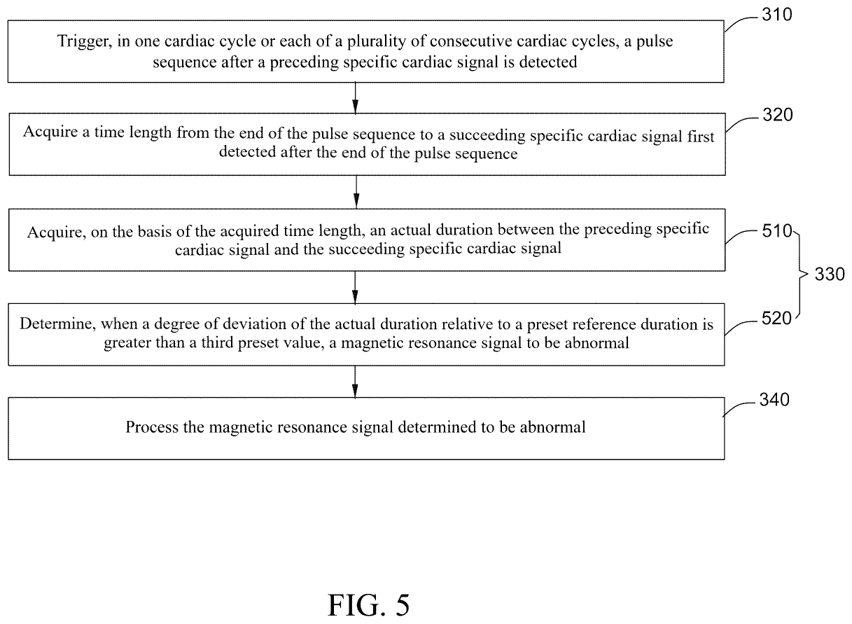

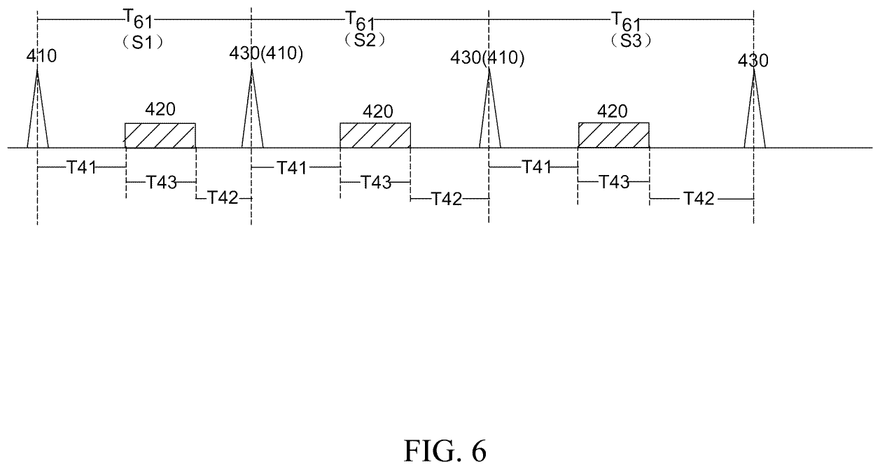

FIG. 5 is a flowchart of a magnetic resonance imaging method according to another embodiment of the present application, and FIG. 6 is a schematic timing diagram of the embodiment shown in FIG. 5. Referring to FIG. 5 and FIG. 6, in some embodiments, determining whether the magnetic resonance signal is abnormal may include steps 510 and 520. In step 510, an actual duration T₆₁ between the preceding specific cardiac signal 410 and the succeeding specific cardiac signal 430 is acquired on the basis of the acquired time length T42 (for example, actual durations of the cardiac cycles S1, S2, etc., mentioned above). Since a time interval between detection of each specific cardiac signal and initiation of the pulse sequence 420 is fixed, and a duration T43 of the pulse sequence 420 is also fixed, the actual duration T₆₁ between the preceding specific cardiac signal 410 and the succeeding specific cardiac signal 430 may be calculated on the basis of the aforementioned time length T42.

The actual duration T₆₁ is used for comparison with a preset reference duration T₀ to determine whether the magnetic resonance signal acquired on the basis of the corresponding pulse sequence 420 is abnormal. This comparison may include, for example, calculating a degree of deviation T_{b} of the actual duration T₆₁ relative to the reference duration T₀. The degree of deviation T_{b} may be, for example, calculated using the following formula (1):

$${T}_{b} = ( {T}_{61} - {T}_{0} ) / {T}_{0} * 100 \% \qquad ( 1 )$$

For example, in step 520, when the degree of deviation T_{b} of the actual duration T₆₁ relative to the reference duration T₀ is greater than a third preset value, the acquired magnetic resonance signal is determined to be abnormal. The reference duration T₀ may be a preferred R-R interval (i.e., an interval between adjacent R waves) determined in advance on the basis of heartbeat detection data of the current subject or other subjects with similar heartbeat characteristics to the current subject, where data acquired at the preferred R-R interval is considered to have a better clinical value.

In step 520, the third preset value may be an upper limit of an acceptable degree of deviation, thus enabling processing of data acquired when the heart rate is overly slow. In other embodiments, a lower limit of the degree of deviation may further be set so that if a specific cardiac signal is detected overly quickly after the end of a pulse sequence, a corresponding magnetic resonance signal is processed.

In some embodiments of the present application, a waiting state may be initiated and timing may be performed after the end of the pulse sequence 420 until the specific succeeding cardiac signal 430 is first detected. On the basis of a result of the timing, the time length T42 can be determined. The aforementioned waiting state refers to the magnetic resonance imaging system not transmitting radio frequency or gradient pulses. The aforementioned timing may be implemented using a system clock, a system controller, a computer system, or a processor in other components of the magnetic resonance imaging system.

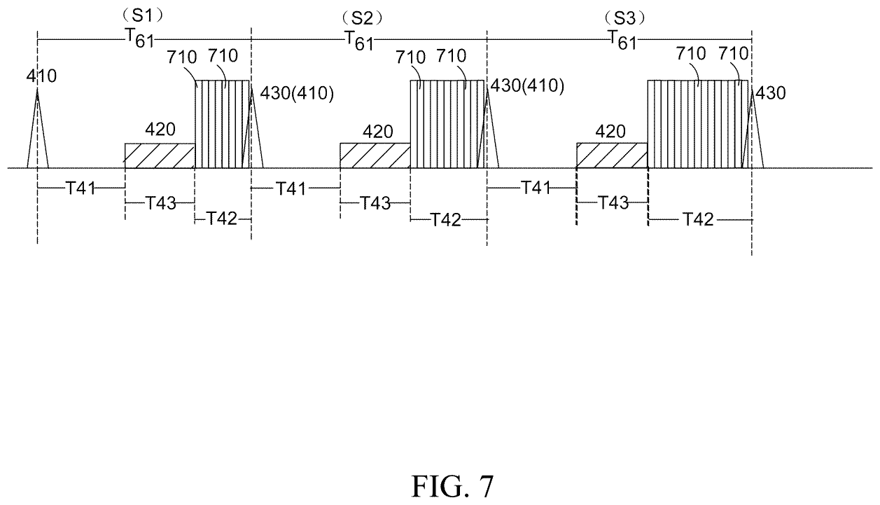

Referring to FIG. 7, in some other embodiments of the present application, delayed pulses 710 may be set that continuously repeat after the end of the pulse sequence 420 until the specific succeeding cardiac signal 430 is first detected after the pulse sequence 420 ends. Further, the delayed pulses 710 between the pulse sequence 420 and the succeeding specific cardiac signal 430 are counted, and the time length T42 may be determined on the basis of a result of the counting. When the pulse sequence 420 is designed, the delayed pulses 710 may be used as an additional part of the pulse sequence 420, that is, the delayed pulses 710 are automatically loaded when the pulse sequence 420 is loaded. Each delay pulse 710 may cover a fixed unit of time, and the time length T42 may be obtained by multiplying the unit of time by the count value of the delay pulses 710.

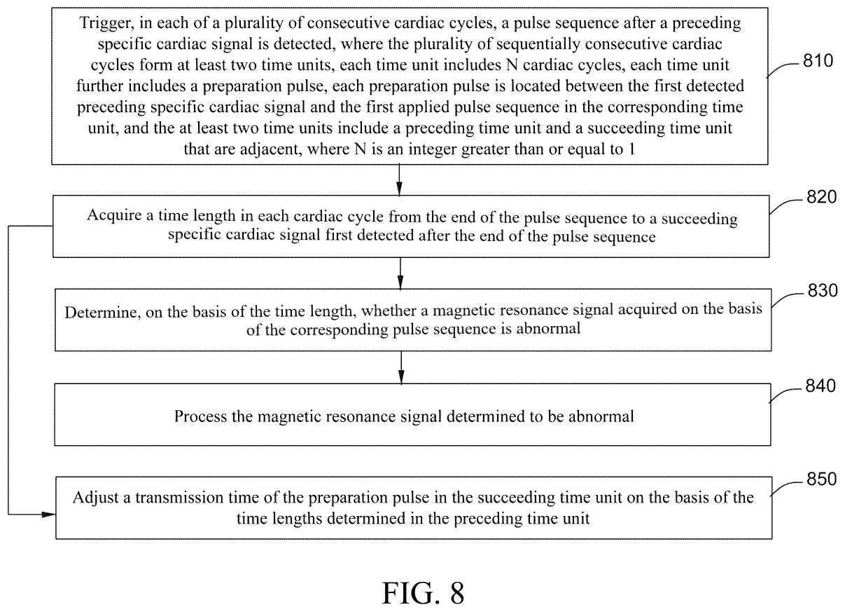

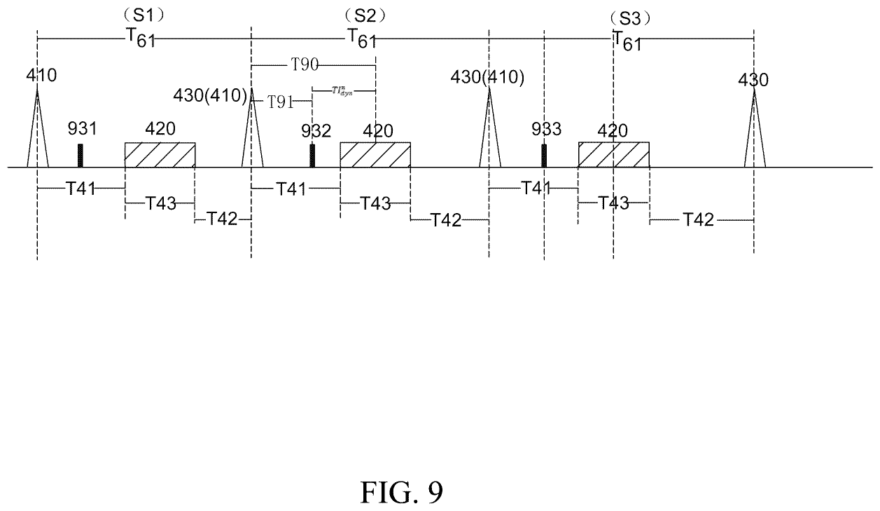

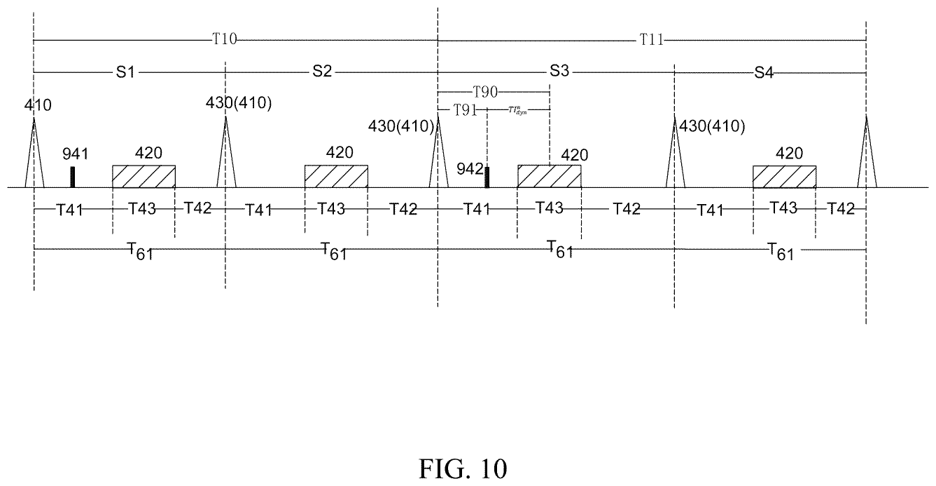

FIG. 8 is a flowchart of a magnetic resonance imaging method according to another embodiment of the present application, and FIG. 9 is an example of a timing diagram of the embodiment shown in FIG. 8. FIG. 10 is another example of a timing diagram of the embodiment shown in FIG. 8. Referring to FIG. 8, FIG. 9, and FIG. 10, in step 810, in each of a plurality of consecutive cardiac cycles, a pulse sequence 420 is triggered after a preceding specific cardiac signal 410 is detected. In step 820, a time length T42 from the end of the pulse sequence 420 to a specific succeeding cardiac signal 430 first detected after the end of the pulse sequence 420 in each cardiac cycle is acquired. In step 830, on the basis of the time length T42, it is determined whether a magnetic resonance signal acquired on the basis of the corresponding pulse sequence 420 is abnormal. In step 840, the magnetic resonance signal that is determined to be abnormal is processed.

In some embodiments, the plurality of consecutive cardiac cycles form at least two time units, each time unit includes N cardiac cycles, each time unit further includes a preparation pulse, and each preparation pulse is located between the first detected preceding specific cardiac signal 410 and the first applied pulse sequence 420 in the corresponding time unit (triggered via the first detected preceding specific cardiac signal 410). The at least two time units include a preceding time unit and a succeeding time unit that are adjacent, where N is an integer greater than or equal to 1.

Using the timing sequence shown in FIG. 9 as an example, N equals 1, meaning that each of cardiac cycles S1, S2, and S3 in FIG. 9 may be used as (or correspond to) a time unit. In other words, each time unit includes one cardiac cycle, and each cardiac cycle (or each time unit) further includes a preparation pulse, for example, preparation pulses 931, 932, and 933 are applied during the cardiac cycles S1, S2, and S3, respectively. The preparation pulse 931 is located after the first detected (and only) preceding specific cardiac signal 410 in the corresponding cardiac cycle S1, and is located before the first (and only) applied pulse sequence 420 in the cardiac cycle S1. The preparation pulse 932 is located after the first detected (and only) preceding specific cardiac signal 410 in the corresponding cardiac cycle S2, and is located before the first (and only) applied pulse sequence 420 in the cardiac cycle S2. The preparation pulse 933 is located after the first detected (and only) preceding specific cardiac signal 410 in the corresponding cardiac cycle S3, and is located before the first (and only) applied pulse sequence 420 in the cardiac cycle S3. The plurality of consecutive cardiac cycles include a preceding cardiac cycle and a succeeding cardiac cycle that are adjacent. For example, the cardiac cycles S1 and S2 may be used as a preceding cardiac cycle and a succeeding cardiac cycle, respectively, and the cardiac cycles S2 and S3 may also be used as a preceding cardiac cycle and a succeeding cardiac cycle, respectively.

Using FIG. 10 as an example, N equals 2, meaning that every two of the cardiac cycles S1, S2, S3, and S4 in FIG. 10 may be used as (or correspond to) a time unit. In other words, each time unit includes two cardiac cycles. For example, FIG. 10 shows two adjacent time units T11 and T12, which are used as a preceding time unit and a succeeding time unit, respectively. The preceding time unit T11 includes the cardiac cycles S1 and S2. A preceding specific cardiac signal 410 in the cardiac cycle S1 is the first detected preceding specific cardiac signal in the preceding time unit T11, and a pulse sequence 420 applied in the cardiac cycle S1 is the first applied pulse sequence in the preceding time unit T11. The succeeding time unit T12 includes the cardiac cycles S3 and S4. A preceding specific cardiac signal 410 in the cardiac cycle S3 is the first detected preceding specific cardiac signal in the succeeding time unit T12, and a pulse sequence 420 applied in the cardiac cycle S3 is the first applied pulse sequence in the succeeding time unit T12. The preceding time unit T11 further includes a preparation pulse 941, which is located between the preceding specific cardiac signal 410 in the cardiac cycle S1 and the pulse sequence 420 in the cardiac cycle S1. The succeeding time unit T12 further includes a preparation pulse 942, which is located between the preceding specific cardiac signal 410 in the cardiac cycle S3 and the pulse sequence 420 in the cardiac cycle S3.

In other embodiments, each time unit may further include more cardiac cycles, which will not be enumerated herein.

As shown in FIG. 9 and FIG. 10, within each time unit, after the first detection of the preceding specific cardiac signal 410 and after second preset delay time T91, a corresponding preparation pulse (e.g., an inversion recovery pulse) is applied. Longitudinal magnetization vectors of all excited tissues are inverted and begin to recover gradually. Then, after each preceding specific cardiac signal 410 detected in the time unit, a pulse sequence 420 is transmitted. After the end (or possibly also before the end) of the pulse sequence 420, a succeeding specific cardiac signal 430 is identified. The succeeding specific cardiac signal 430 and the pulse sequence 420 that was initiated therebefore may have a time length T42 therebetween, or the two may overlap. Each succeeding specific cardiac signal 430 may be used as a preceding specific signal 410 first detected in a succeeding time unit.

The aforementioned pulse sequence 420 may be determined according to clinical needs. For example, the pulse sequence may be part of a myocardial delayed enhancement sequence, which may include, for example, a non-phase-sensitive myocardial delayed enhancement sequence or a phase-sensitive myocardial delayed enhancement sequence, and the myocardial delayed enhancement sequence may be a segmented acquisition sequence with multiple excitations (e.g., acquiring only a portion of a layer of k-space per cardiac cycle) or a single excitation sequence (acquiring one layer of the k-space at a time). The pulse sequence 420 as part of the myocardial delayed enhancement sequence is used for acquisition of a magnetic resonance signal. For example, this part may include a gradient echo sequence (GRE) or an echo planar imaging sequence (EPI). The gradient echo sequence may include a fast gradient echo sequence (FGRE), a spoiled gradient recalled echo sequence (SPGR), a rapid balanced steady-state precession imaging (B-SSFP), etc., which will not be enumerated herein. In some embodiments, other parts of the aforementioned myocardial delayed enhancement sequence may include, for example, a preparation pulse or a delayed pulse (not shown in the figure) mentioned above. Alternatively, other parts of the aforementioned myocardial delayed enhancement sequence may be, for example, sequences for magnetic resonance signal acquisition implemented in other cardiac cycles, as shown in FIG. 10. Two pulse sequences 420 in the time unit T11 and the corresponding preparation pulse 941 constitute a complete myocardial delayed enhancement sequence. This sequence may be, for example, a phase-sensitive myocardial delayed enhancement sequence. In each time unit, a first pulse sequence 420 is used to acquire main image data and a second pulse sequence 420 has a smaller flip angle than the first pulse sequence 420 and is used to acquire reference image data. A final image may be generated using both the reference image data and the main image data. In FIG. 9, the preparation pulse and the pulse sequence 420 in each cardiac cycle form a complete myocardial delayed enhancement sequence, which may be, for example, a non-phase-sensitive myocardial delayed enhancement sequence.

In some optional embodiments, when a magnetic resonance signal acquired via the pulse sequence 420 in the cardiac cycle S2 of FIG. 10 is determined to be abnormal, the preparation pulse 942 may not be transmitted in the next cardiac cycle S3, but instead, the pulse sequence 420 may be directly triggered to acquire a reference image.

In one example, the preparation pulse 910 may be an inversion recovery pulse, one clinical application of which includes suppressing a specific tissue in magnetic resonance imaging to enhance other tissues, thereby achieving an enhanced imaging effect.

To facilitate understanding, the principle of the aforementioned myocardial delayed enhancement sequence will be briefly explained below.

During cardiac scanning, a contrast agent (for example, a gadolinium contrast agent) may be intravenously injected into a subject, and the heart is scanned after waiting for 10-30 minutes. Normal myocardium is characterized by “fast in and fast out”, and the contrast agent is substantially washed out 10-30 minutes after the contrast agent is injected. Delayed enhancement is displayed as a low signal. For abnormal myocardium (fibrosis, scar, infarction), 10-30 minutes after the contrast agent is injected, the myocardial function is lost, the contrast agent washout is impaired, and it is detected by scanning that there is residual contrast agent. Consequently, a longitudinal relaxation time (T1) is significantly shortened, and a high signal is presented relative to normal myocardial tissue. In this way, the normal myocardial tissue and abnormal myocardial tissue are scanned 10-30 minutes after the injection of the contrast agent, and a contrast is formed and may be reflected in an image. The normal myocardial tissue has a relatively long longitudinal relaxation time T1, so that longitudinal relaxation recovery is slow. The abnormal myocardium loses activity and cannot clear the contrast agent, resulting in a relatively short longitudinal relaxation time T1 for infarcted myocardial tissue, so that longitudinal relaxation recovery is fast. The abnormal myocardium exhibits a high signal due to a short relaxation time T1, and thus enhanced (highlighted) display of the abnormal myocardium is implemented.

Similar to a fat inhibition sequence or a free water inhibition sequence, an inversion recovery pulse causes different zero-crossing points to pass through different tissues, thus allowing selective inhibition of certain tissues. By using the inversion recovery pulse, a magnetization vector may be inverted, and an image signal may be recovered from −Mz to MZ. If data is acquired in response to zero-crossing of normal myocardium, an image with maximum contrast for abnormal myocardium can be obtained.

In some examples, by scanning a group of a plurality of contrast images with different inversion times (TI) and determining by comparison which myocardial tissue has the lowest signal in the group of images, a zero-crossing (zero-return) time value TIₙᵤₗₗ of normal myocardium corresponding to the image can be selected as the zero-crossing time of normal myocardium. Within a cardiac cycle, TIₙᵤₗₗ indicates the time from an inversion recovery pulse to the center of filling K, or the time from the inversion recovery pulse to a signal acquisition center moment. By identifying an appropriate TIₙᵤₗₗ of normal myocardium and transmitting an inversion recovery pulse according to this TIₙᵤₗₗ, a normal myocardial signal is suppressed-“made hypointense”, and compared to the normal myocardium, an abnormal myocardial signal of a residual gadolinium contrast agent will be significantly highlighted and enhanced.

Further referring to FIG. 8, one difference from the embodiment shown in FIG. 2 is that step 810 is further included, in which a transmission time of the preparation pulse in the succeeding time unit is adjusted on the basis of the time length T42 determined in the preceding time unit. This will be described in detail below.

In some embodiments, an apparent longitudinal relaxation time T1 of a first tissue to be suppressed may be estimated on the basis of a zero-crossing time of the first tissue to be suppressed. Continuing with the example of the first tissue being normal myocardium, the apparent longitudinal relaxation time T1 is not a true T1 of a normal myocardial tissue, but is estimated on the basis of a zero-crossing time of a normal myocardial tissue.

For example, the zero-point time TIₙᵤₗₗ of the first tissue and the apparent T1 of the first tissue (TI_{amyo}) satisfy the following formula (2):

$${TI}_{null} = {TI}_{amyo} \cdot [ ln 2 - ln ( 1 + {e}^{\frac{- {TR}_{eff}}{T {1}_{amyo}}} ) ] \qquad Formula ( 2 )$$

TR_{eff}=#RR×1000×60/HR, where HR represents a current or estimated cardiac rhythm of the subject in beats per minute (BPM), and #RR is the number of heartbeats in the preceding cardiac cycle (or preceding time unit, which includes one or more cardiac cycles). TIₙᵤₗₗ and TR_{eff} are substituted into Formula (1) to perform inverse solution, to obtain the apparent longitudinal relaxation time T1.

Furthermore, a transmission time of the preparation pulse in the succeeding cardiac time unit may be adjusted according to the apparent longitudinal relaxation time TI_{amyo} and on the basis of one or more time lengths T42 determined in the preceding time unit, for example, a second preset delay time T91 may be adjusted. Specifically, a position of an inversion recovery pulse in the succeeding time unit may be determined according to the apparent longitudinal relaxation time TI_{amyo} and duration

$${TR}_{eff}^{n - 1}$$

of the preceding time unit, for example, a transmission time of the inversion recovery pulse in the succeeding time unit may be determined. The duration

$${TR}_{eff}^{n - 1}$$

of the preceding time unit may be determined on the basis of one or more time lengths T42 therein. Specifically, the duration

$${TR}_{eff}^{n - 1}$$

of the preceding time unit may be determined on the basis of the first preset delay time T41 of each cardiac cycle (e.g., an empirical value), the time length T42 determined in the embodiments of the present application, and a duration T43 of the pulse sequence 420 (which is a known value). For example, in FIG. 9, the duration

$${TR}_{eff}^{n - 1}$$

of the preceding time unit is the duration T₆₁ of a corresponding cardiac cycle; in FIG. 10, the duration

$${TR}_{eff}^{n - 1}$$

of the preceding time unit T11 is a sum of the durations T₆₁ of the corresponding cardiac cycles S1 and S2.

In some embodiments, an inversion time

$${TI}_{dyn}^{n}$$

of the succeeding time unit may be determined according to the following formula (3) or (4).

$${TI}_{dyn}^{n} = {TI}_{amyo} \cdot [ ln 2 - ln ( 1 + {e}^{\frac{- {TR}_{eff}^{n - 1}}{{T}_{{1}_{amyo}}}} ) ] \qquad Formula ( 3 )$$

$${TI}_{dyn}^{n} = {TI}_{amyo} \cdot [ ln 2 - ln ( 1 + {e}^{\frac{- {TR}_{eff}^{avg}}{{T}_{{1}_{amyo}}}} ) ] \qquad Formula ( 4 )$$

A difference from formula (3) is that

$${TR}_{eff}^{avg}$$

is not equal to duration

$${TR}_{eff}^{n - 1} ,$$

but instead is determined on the basis of the duration

$${TR}_{eff}^{n - 1} .$$

$${TR}_{eff}^{avg}$$

may be equal to a weighted average of X

$${TR}_{eff}^{n - 1} ,$$

where S is an integer greater than of equal to 2.

Using the time unit S2 in FIG. 9 or the time unit T12 in FIG. 10 as an example, the inversion time

$${TI}_{dyn}^{n}$$

is a time length between an inversion recovery pulse transmitted in the succeeding time unit and a signal acquisition center moment of the first pulse sequence in the succeeding time unit. Therefore, the position of the inversion recovery pulse in the succeeding time unit may be determined according to this inversion time

$${TI}_{dyn}^{n} .$$

Within the corresponding cardiac cycle, a transmission moment of the inversion recovery pulse is

$$T 90 - {TI}_{dyn}^{n} ,$$

where T90 is the signal acquisition center moment, which may be determined on the basis of the first preset delay time T41 and the duration T43 of the pulse sequence 420, thereby allowing for dynamic adjustment of the second preset delay time T91, and further adjusting the transmission moment of the preparation pulse.

By dynamically adjusting the transmission moment of the preparation pulse, a specific tissue may be enhanced to a greater extent, such as highlighting abnormal myocardium in images.

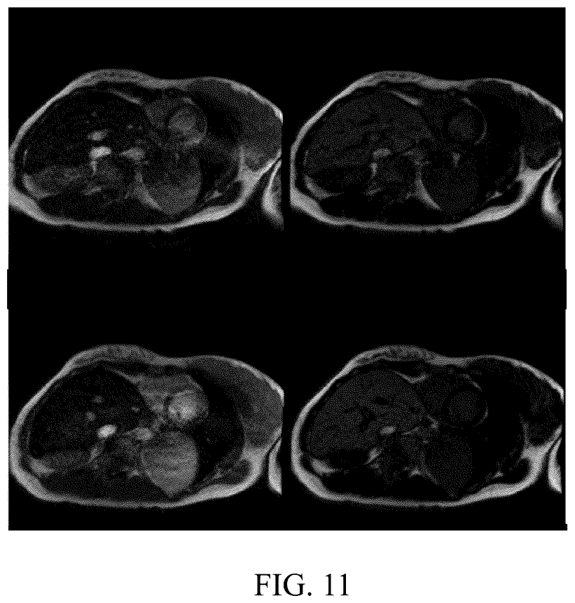

FIG. 11 shows images obtained by performing magnetic resonance scans on a subject with arrhythmia using an embodiment of the present application and the prior art. The two images on the left are images obtained using a conventional method, and the two images on the right are images obtained using the method of the embodiment of the present application. The comparison shows that the use of the method of the embodiment of the present application reduces artifacts in the images. Embodiments of the present application further provide a magnetic resonance imaging system. The magnetic resonance imaging system may include at least a part of the system shown in FIG. 1, and repeated parts will not be described again.

In some embodiments, the magnetic resonance imaging system includes a scanning unit and a controller. The controller may be configured to control the scanning unit to acquire a magnetic resonance signal and perform the magnetic resonance imaging method described in any embodiment of the present application.

In some embodiments, the MRI system controller 130, the image processor 128, and the physiological acquisition processor 155 shown in FIG. 1 may be provided individually or jointly to the controller, or the controller may be integrated with the MRI system controller 130, the image processor 128, the physiological acquisition processor 155, or the like. The controller may include a computer processor and a storage medium on which a predetermined data processing program that is to be executed by the computer processor is recorded. For example, the storage medium may store a program used to implement scanning processing (e.g., waveform design/conversion, etc.), image reconstruction, image processing, etc. For example, the storage medium may store a program used to implement the scan sequence generation method of the embodiments of the present application, specific implementations of which are as described above and will not be repeated herein.

The above storage medium may include, for example, a ROM, a floppy disk, a hard disk, an optical disk, a magneto-optical disk, a CD-ROM, or a non-volatile memory card.

An embodiment of the present application further provides a computer-readable program. When the program is executed in an apparatus or a magnetic resonance imaging system, the program causes a computer to execute, in the apparatus or the magnetic resonance imaging system, the method described in the above embodiments.

An embodiment of the present application further provides a storage medium having a computer-readable program stored thereon. The computer-readable program causes a computer to execute, in an apparatus or a magnetic resonance imaging system, the method described in the above embodiments.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a magnetic disk, an optical disc, a DVD, a flash memory, etc.

The method/apparatus described with reference to the embodiments of the present application may be directly embodied as hardware, a software module executed by a processor, or a combination of the two. For example, one or more of the functional block diagrams and/or one or more combinations of the functional block diagrams shown in the figures may correspond to either software modules or hardware modules of a computer program flow. The foregoing software modules may respectively correspond to the steps shown in the figures. The foregoing hardware modules may be implemented, for example, by consolidating the foregoing software modules by using a field-programmable gate array (FPGA).

The software modules may be located in a RAM memory, a flash memory, a ROM memory, an EPROM memory, an EEPROM memory, a register, a hard disk, a removable disk, a CD-ROM, or any storage medium in other forms known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, then the software modules may be stored in the MEGA-SIM card or the large-capacity flash memory apparatus.

One or more of the functional blocks and/or one or more combinations of the functional blocks shown in the drawings may be implemented as a general-purpose processor, a digital signal processor (DSP), an application-specific integrated circuit (ASIC), a field-programmable gate array (FPGA) or other programmable logic devices, a discrete gate or transistor logic device, a discrete hardware assembly, or any appropriate combination thereof, which is used for executing the functions described in the present application. The one or more functional blocks and/or the one or more combinations of the functional blocks shown in the drawings may alternatively be implemented as a combination of computing devices, such as a combination of a DSP and a microprocessor, a plurality of microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

What is claimed is:

1. A magnetic resonance imaging method, comprising:
triggering, in one cardiac cycle or each of a plurality of consecutive cardiac cycles, after a preceding specific cardiac signal is detected, a pulse sequence for acquiring a magnetic resonance signal;
acquiring a time length from the end of the pulse sequence to a succeeding cardiac signal first detected after the end of the pulse sequence;
determining, on the basis of the time length, whether the magnetic resonance signal acquired on the basis of the pulse sequence is abnormal; and
processing the abnormal magnetic resonance signal.

2. The method according to claim 1, wherein determining whether the magnetic resonance signal is abnormal comprises:
determining, when the time length is greater than a first preset value, the magnetic resonance signal to be abnormal.

3. The method according to claim 1, wherein determining whether the magnetic resonance signal is abnormal comprises:
determining, when the time length is less than a second preset value, or when another specific cardiac signal is detected during the pulse sequence, the magnetic resonance signal to be abnormal.

4. The method according to claim 1, wherein determining whether the magnetic resonance signal is abnormal comprises:
acquiring, on the basis of the time length, an actual duration between the preceding specific cardiac signal and the succeeding specific cardiac signal; and
determining, when a degree of deviation of the actual duration relative to a preset reference duration is greater than a third preset value, the magnetic resonance signal to be abnormal.

5. The method according to claim 1, wherein acquiring the time length comprises:
initiating a waiting state after the end of the pulse sequence and performing timing until the succeeding specific cardiac signal is detected.

6. The method according to claim 1, wherein acquiring the time length comprises:
setting delayed pulses that continuously repeat after the end of the pulse sequence until the succeeding specific cardiac signal is detected; and
counting the delayed pulses from the end of the pulse sequence to the succeeding specific cardiac signal, and determining the time length on the basis of a result of the counting.

7. The method according to claim 1, wherein processing the magnetic resonance signal comprises: discarding or correcting the magnetic resonance signal acquired via the corresponding pulse sequence.

8. The method according to claim 1, wherein
the plurality of consecutive cardiac cycles form at least two time units, each time unit comprises N cardiac cycles, each time unit further comprises a preparation pulse, each preparation pulse is located between the first detected preceding specific cardiac signal and the first applied pulse sequence in the corresponding time unit, and the at least two time units comprise a preceding time unit and a succeeding time unit that are adjacent, where N is an integer greater than or equal to 1; and the method further comprises:
adjusting a transmission time of the preparation pulse in the succeeding time unit on the basis of the N time lengths determined in the preceding time unit.

9. The method according to claim 1, wherein the pulse sequence is a non-phase-sensitive myocardial delayed enhancement sequence or a phase-sensitive myocardial delayed enhancement sequence.

10. The method according to claim 1, wherein the preceding specific cardiac signal and the succeeding specific cardiac signal are R waves in an electrocardiogram waveform acquired by performing electrocardiogram a subject.

11. A magnetic resonance imaging system, comprising:
a scanning unit; and
a controller, configured to control the scanning unit to acquire a magnetic resonance signal and perform the magnetic resonance imaging method comprising:
triggering, in one cardiac cycle or each of a plurality of consecutive cardiac cycles, after a preceding specific cardiac signal is detected, a pulse sequence for acquiring a magnetic resonance signal;
acquiring a time length from the end of the pulse sequence to a succeeding cardiac signal first detected after the end of the pulse sequence;
determining, on the basis of the time length, whether the magnetic resonance signal acquired on the basis of the pulse sequence is abnormal; and
processing the abnormal magnetic resonance signal.
